(19) United States
(12) Patent Application Publication
(10) Pub. No.: US 2017/0205409 A1
(43) Pub. Date: Jul. 20, 2017

(54) HPV16 Antibodies as Diagnostic and Prognostic Biomarkers in Pre-Invasive and Invasive Disease

(71) Applicants: ARIZONA BOARD OF REGENTS ON BEHALF OF ARIZONA STATE UNIVERSITY, Scottsdale, AZ (US); The United States of America, as Represented by the Secretary, Department of Health and Human Serv, Bethesda, MD (US)

(72) Inventors: Karen Anderson, Scottsdale, AZ (US); Elizabeth Unger, Atlanta, GA (US)

(21) Appl. No.: 15/129,370

(22) PCT Filed: Mar. 19, 2015
(86) PCT No.: PCT/US15/21563

§ 371 Date: Sep. 26, 2016

Related U.S. Application Data
(60) Provisional application No. 61/971,425, filed on Mar. 27, 2014.

(51) Int. Cl. G01N 33/569; G01N 33/574

(52) U.S. Cl. CPC G01N 33/56983; G01N 33/57411; G01N 2333/025

(57) ABSTRACT

Methods and systems for detection of HPV mediated cervical or oropharyneal cancer are provided. The methods include contacting a fluid sample from a patient with multiple antibodies to HPV16 early gene proteins and comparing patterns of HPV16 antibody bound to said early gene proteins with a control associated with cervical or oropharyneal cancer (FIG. 1).

### CROSS-REFERENCE TO RELATED APPLICATIONS

This application claims priority to U.S. Provisional Patent Application No. 61/971,425 filed on Mar. 27, 2014.

### STATEMENT OF GOVERNMENT RIGHTS

This invention was made with government support under U01 CA117374 awarded by the National Institute of Health. The government has certain rights in the invention.

### TECHNICAL FIELD

This invention relates to methods and materials involving biomarkers for diagnostic and prognostic use with HPV-associated diseases.

### BACKGROUND

The detection of the humoral immune response is essential for the diagnosis and prognosis of infectious disease and autoimmunity, and may also provide biomarkers for the detection of cancer. Several proteomic multiplexed immunoassays have been developed to facilitate the detection of these antibodies. The slide-based assays, in particular, are excellent discovery tools for the detection of antibodies, but require specialized high-throughput equipment not generally found in routine immunology laboratories.

Human papillomavirus (HPV) is the most common sexually acquired infection, with estimates that up to 75% of sexually active people are infected at some time in their lifetime. Genital infection with HPV is usually acquired shortly after sexual debut, and prevalence is highest in adolescents and young adults. In most cases infections are transient and asymptomatic, and prevalence generally decreases with age. Persistent genital infection is more likely to be associated with neoplastic progression, with invasive cancer occurring many years (generally decades) after infection. Infection with HPV16 and 18 has been clearly associated with oropharyngeal cancer (OPC), cervical cancer, anal cancers, and other malignancies. Indeed, it is well established that most cases of OPCs in the Western world are linked to HPV infection and the numbers are rising.

Acute HPV infections induce humoral immune responses, primarily to the HPV-derived latent protein L1. Abs to L1 capsid protein are induced after viral infection and persist for years. Abs to both E6 and E7 have been detected at low levels in both senrum and cervical vaginal secretions of cervical cancer patients and in the sera of OPC patients. Abs to HPV16 E6 and HPVI6 E7 develop later in the course of ICC, and have been shown to correlate with disease outcome. Studies of sera collected prior to the diagnosis of cervical cancer have shown that the presence of E6 and E7-specific antibodies is associated with an increased relative risk for cervical cancer of 2.7, and can be detected up to 5 years prior to diagnosis. It is not known if quantitative or qualitative antibody levels in serum and/or cervical mucous would predict clearance versus persistence and progression.

### SUMMARY

There is a need for a biomarker that can serve as a diagnostic and prognostic detector for HPV-associated diseases, including invasive cervical cancer (ICC) and oropharyngeal cancers (OPC). HPV DNA testing is significantly more sensitive than the current screening method for ICC, and with the advent of the HPV-vaccine, there is a need for screening for vaccine-missed cervical cancers that are cost-effective and specific.

The purpose of this disclosure is to determine the discriminating properties of HPV16-specific early gene antibodies as biomarkers for the early diagnosis of ICC and OPC, as well as biomarkers for prognosis and risk assessment. The inventors have found that the patterns of HPV16 antibodies were markedly different between ICC and OPC, and in ICC are strongly associated with cervical disease progression but not HPV16 infection. This data support the hypothesis that HPV antibody responses and antibody signatures are specific biomarkers of HPV-associated malignancies and can be applied to early detection, prognosis, and risk assessment.

Unless otherwise defined, all technical and scientific terms used herein have the same meaning as commonly understood by one of ordinary skill in the art to which this disclosure belongs. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 depicts specific detection of multiple HPV16 antibodies in patients with cervical disease. HPV16 proteins were expressed as GST fusion proteins and captured on Luminex beads. The MFI ratio (MFI (HPV)/MFI (p21-GST) of IgG detected in sera is shown. Serum IgG responses were measured in patients with CIN 0/I, CIN II/III, and invasive cervical cancer. HPV16-specific Abs to E1, NE2, E4, E6 and E7 proteins are detected in patients with invasive cervical cancer, compared to women with preinvasive disease CIN II/II or CIN 0/I controls. There is no significant difference in individual serology between CIN 0/I and CIN II/III.

FIG. 2 shows the specificity of serologic assay for HPV16 IgG. A) Selective detection of HPV16 antibodies in sera of HPV16+ cases. Subset analysis was performed on 54 cases of invasive cervical cancer, for which HPV16 tumor status was known (HPV16+, n=34; HPV16−, n=20).

FIG. 3 illustrates a comparison of HPV16 Abs in HR HPV+ oropharyngeal and HPV16+ cervical cancers. HPV16 Ab levels were measured in the blood of invasive cervical (n=34) and oropharyngeal cancer patients (OPC, n=5O). HPV16 Abs were more strongly detected in OPC for HPV16 E1, NE2, CE2, E6, and E7 (p<0.0001).

FIG. 4 depicts the unsupervised hierarchical clustering of HPV16-specific Abs in HPV16+ invasive cervical cancer (ICC, n=34) and oropharyngeal cancer (HPVOPC, n=50) patient sera. ICC patients either have primarily HPV16 E7 Abs (group I) or no Abs (group II). The majority of patients with HPVOPC (group III) have multiple HPV-specific Abs, including E1, E2, E6, and E7. Intensity is shown in logarithmic scale.

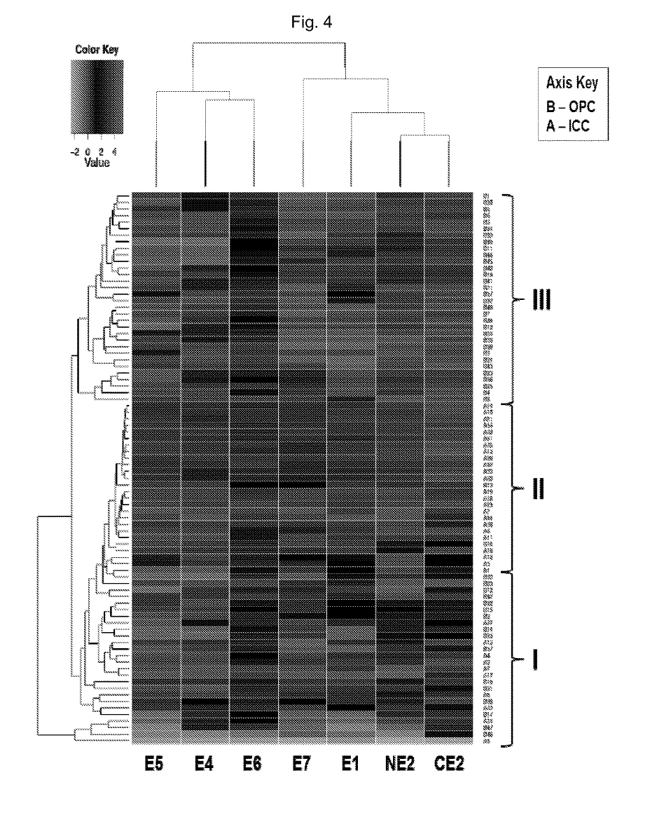

### DETAILED DESCRIPTION

Embodiments described herein relate to recently adapted novel protein array technology for the detection of antibodies in sera. Full length cDNA's encoding HPV16 antigens are expressed as c-terminal GST fusion proteins using mammalian in vitro transcription/translation, and captured onto Luminex bead arrays (RAPID bead array ELISA. Using sera from patients with OPC, we have specifically detected antibodies to multiple HPV16-derived antigens, including E1, E2, E4, E6, and E7 antibodies. We also detected variability in the patterns of immune responses within a clinically homogeneous cohort of patients, suggesting that there are biologic differences in the immune recognition of this virus in OPC patients.

To determine whether immune responses to HPV are potential biomarkers for detection and prognosis in pre-invasive and invasive cervical disease, we have used RAPID bead array ELISAs for the detection of serum and cervical secretion IgG and IgA Abs to HPV16-derived antigens. To evaluate the potential utility of these antibodies as biomarkers, we compared these results with the detection of serum IgG Abs in an expanded cohort of patients with oropharyngeal cancer. Here, we demonstrate that the breadth and quantity of HPV16 IgG to early genes increase with progressive cervical disease, and early-gene Abs are specific biomarkers of invasive HPVI6-associated carcinomas.

Further, sera from patients with OPC have distinct patterns of HPV16-specific Abs compared to ICC, suggesting differences in the pathophysiology of viral antigen expression or immune surveillance between these two anatomic sites.

In other embodiments, systems for diagnosing human papillomavirus (HPV) mediated cancer are described. For example, a system may include a substrate (such as a peptide chip) having multiple antibodies to HPV16 early gene proteins coupled thereto, a visualization agent (e.g., one or more labeled secondary antibodies), and a control with a binding pattern associated with a HPV-mediated cancer for comparing visualized patterns of HPV16 antibody bound to the early gene proteins with the control associated with a HPV-mediated cancer.

The invention will be further described in the following examples, which do not limit the scope of the invention described in the claims.

### EXAMPLES

### Example 1

Sera used in the cervical disease analysis were selected from an Early Detection Research Network (EDRN) and Centers for Disease Control and Prevention biorepository collected from women attending colposcopy clinics at urban public hospitals in Atlanta, Ga., Detroit, Mich. or Galveston, Tex. The set consisted of CIN 0II (n:121) and CIN II/III (n:162) patient sera, representing patients who present to colposcopy clinics. Of these, a subset of CIN 0/I (n:33) and CIN IVIII (n=52) were matched to age, race, and HPV16 status. Archived anonymized sera from 95 women with invasive cancer were included for analysis. OPC cancer patient sera were obtained from the Dana Farber Cancer Institute, Johns Hopkins Medical Center, and Mt. Sinai School of Medicine. All samples were obtained prior to treatment of cancer, and were selected retrospectively. Demographics of the study populations are shown in Table 1. In all studies, samples were collected using a standardized sample collection protocol and stored at −80° C. until use. Written informed consent was obtained from all subjects under institutional review board approval.

Cervical secretions were selected from the same EDRN biorepository and were available for 74 women contributing sera for the preinvasive comparison and for 13 women with invasive cervical cancer. Methods of collection and processing has been previous described. Briefly, cervical secretions were collected by absorption into Weck-Cel′sponges (Xomed Surgical Products, Jacksonville, Fla.) that were snap frozen and stored at −80° C. until extracted with M-PER′ extraction reagent.

HPV16 genes were obtained by nested PCR using gene-specific primers from HPV16 plasmid DNA (American Type Culture Collection, Manassas, Va.) as described. The PCR products were inserted into pDONR221 vector per manufacturer's instructions (Invitrogen, Carlsbad, Calif.), and were converted to the pANTT_GST vector (http://dnasu.asu.edu/DNASU/Homejsp) for maximal protein expression (24). SeroMAP carboxylated microspheres (Luminex Corporation, Austin, Tex.) were coupled at a ratio of 5 mg anti-GST antisera (GE Healthcare, Piscat away, NJ) to 1 million beads. Each HPV gene was expressed as GST-fusion proteins using T7 reticulocyte lysate (Promega Corporation, Madison, Wis.) per manufacturer's recommendations with 500 ng DNA. p21-GST was expressed as a negative control protein. HPVI6 E2 was expressed as N-terminal NE2 (bp#2755-3303) and Cterminal CE2 (bp#3304-3852) proteins which markedly improved both protein expression and Ab detection. Bead array ELISAs were performed essentially as described.

The in vitro transcriptior/translation (IVTT) products were each captured onto microspheres, pooled, and blocked with 10% each of normal sera from mouse, rabbit, goat, and rat, 0.5% polyvinyl alcohol (PVA, Sigma-Aldrich, St. Louis, Mo.), 0.8% polyvinylpynolidone (PVP, Sigma-Aldrich, St. Louis, Mo.), and 2.5% Superchemiblock (Millipore, Billerica, Mass.) in PBS-I % BSA. Sera were diluted 1:80, and cervical mucous was diluted at 1:5 in blocking buffer and incubated with the beads overnight. Biotin-conjugated goat anti-human IgG or IgA antibody (Jackson ImmunoResearch Laboratories, Inc., West Grove, Pa.) and Strepavidin-R-PE (Molecular Probes, Inc., Eugene, Oreg.) were used for detection of IgG. To establish ELISA cut-off values, an MFI ratio>(the average +3 standard deviations) of 50 healthy control samples was designated positive. These levels were E1:5.4, NE2: 8.5, CE2:6.8, E4: 2.3,E.5: 4.2, E6: 9.0, E7: 6.6, L1: 9.5, and L2: 8.0.

HPV DNA was detected in extracts of exfoliated cervical cells collected in PreservCyt media as previously described. Briefly, 16 ml of the PreservCyt collection media was extracted using MasterPure Complete DNA and RNA purification kit (Epicentre, Madison, Wis.). HPV detection and typing was performed using the Roche linear assay that detects 22 high risk and 15 low risk types.

HPV16 VLPs prepared from baculovirus expression in insect cells were used in a modified direct ELISA to detect IgG. A reference serum sample calibrated against the HPV16 International Standard serum (IS-16, NIBSC, UK) for antibodies (IU/ml) was assayed on each plate. Test samples were diluted 3.16 fold at 1:10, 1:31.6, and 1:100 for testing and antibody titers determined using the parallel line analysis method. Pooled adult human sera that had low and negative reactivity to HPV16 as determined by in-house blocking assay or cLIA were used as positive and negative controls. The pooled negative serum was negative for antibodies to HPV16, 18, 6 and 11, and was used to generate the cut-off value in reference to the IS-16. Antibody titers were calculated for each sample in reference to the HPV16 International Standard serum (NIBSC, UK). A total of 77 sera from pre-invasive cervical disease were tested by HPV16 VLP-IgG ELISA for comparison with the Bead Array ELISA.

The secreted alkaline phosphatase (SEAP) HPV16 pseudovirion neutralization (PsVN) assay measures functional L1IL2-specific antibodies and was performed as described with a few modifications. Serum samples were diluted 2-fold in neutralization buffer [DMEM without phenol red with 1% Non-essential amino acids, 1% Glutamax, 10% fetal bovine serum, 1% antibiotic-antimycotic, and 1% Hepes (pH 7.5)]. The final sample dilutions ranged from 1:20 to 1:10240 for serum and tested on both HPV16 and BPV1 pseudovirions. Positive titers were calculated as the reciprocal of the highest dilution that showed a 50% neutralization of SEAP activity compared to that of the HPV16 pseudovirus in neutralization buffer alone. Serum samples were considered positive if titers were 40 or above and had a four-fold difference with that of BPV 1 neutralization titer for the same sample. A total of 66 sera from pre-invasive cervical disease were tested for comparison with the Bead Array ELISA.

HPV16 Abs were measured as median fluorescence intensity (MFI) using the Luminex200 IS 2.3 software. Fifty events were counted for each bead region. Comparisons were performed using Mann-Whitney nonparametric analysis (GraphPad Prism version 5.0c, San Diego, Calif.). Cohen's Kappa statistic evaluated the agreement between HPV16 PsVN assay, VLP-IgG ELISA and the bead array ELISA. McNemar's test was also performed to evaluate the likelihood of one test being more likely to be positive than the other. Odds ratios (ORs) for predicting the likelihood of having antibody positive response were also estimated using logistic regression models. For each estimate, 95% percent confidence intervals (CIs) were computed. Statistical significance for these tests was achieved at p<0.05 level. Data analyses were performed in SPSS statistics Version 17.0 (SPSS Inc.), using VassarStats Website for Statistical Computation, or R Version 2.9.2.

Our primary goal was to determine if there were quantitative and qualitative differences in HPV16-specific Abs between healthy control women with CIN 0/I (n=121) and women with CIN II/III (n=162), for use as biomarkers to improve the specificity of detection and risk assessment in addition to HR HPV DNA for CIN II/III. Our secondary goal was to determine the specificity of HPV16 antibody biomarker frequencies for different HPV-associated malignancies. Sera and cervical secretions from patients with CIN 0/I, CIN II/III, and ICC were retrospectively selected from the NCI EDRN biorepository at the Centers for Disease Control and Prevention. All samples were restrospectively obtained from women undergoing colposcopy, designed to be representative of a screening colposcopy clinic.

The CIN 0/1 and II/III samples were evenly matched for age (Table 1) and partially matched for HR HPV. There was a higher proportion of CIN II/III cases than CIN 0/1 that were HR HPV+(95.7% vs 59.5%, in particular HPV16+) and a substantial number of patients in both groups had cervical infection with at least 2 types (Table 1).

In the US where cervical Pap screening is common, invasive cervical (ICC) cancer and invasive OPC are both relatively uncommon malignancies. We retrospectively selected banked plasma or sera from patients with ICC; no consistent differences were noted between plasma and sera and the results were pooled for analysis. These samples were obtained over the previous 10 years. As expected, these patients were older than women with CIN II/III (mean 50.1 yrs vs. 29.3 yrs, Table 1), reflecting the time delay of cervical carcinogenesis. Over 50% of the ICC cases were HPV16+. A significant percentage (19.4%) of invasive cervical cases was reported as HPV negative. The HR HPV+ OPC cases, also mixed sera and plasma, were obtained after clinical diagnosis, prior to onset of therapy. As expected, the majority of these cases were male (93.9%).

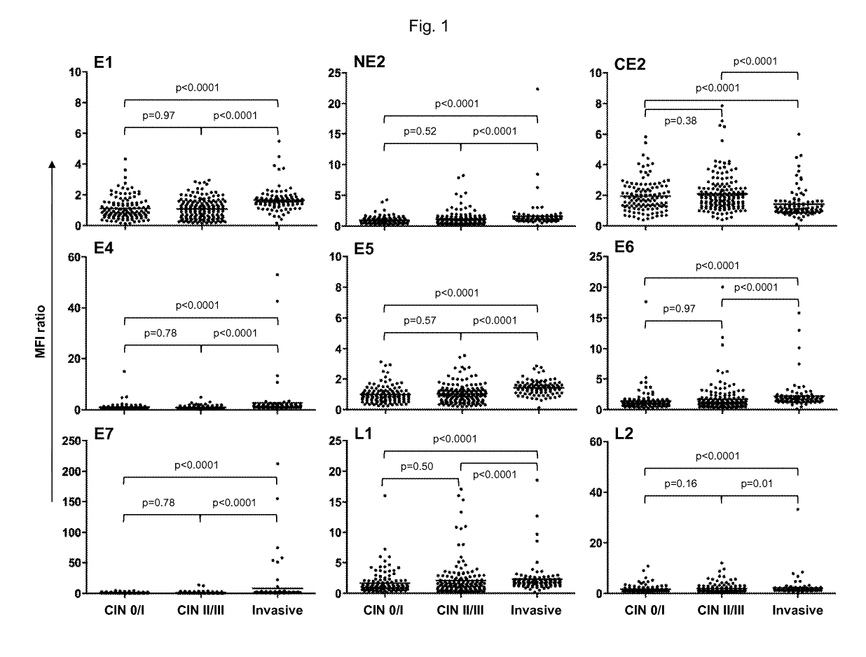

Serum IgG Abs to HPV16 antigens were measured in CIN 0/1, CIN II/III, and invasive cervical cancer patient blood by bead array ELISA (FIG. 1). To control for non-specific and GST-specific autoantibody background, the ratio of MFI for individual HPV-specific Abs to the MFI for the control p21-GST antigen is shown (Table 2a). At least one HPV16 E1, E2, E6, or E7 Ab was detected in the sera of 9/34 (26%) HPV16+ ICC cases, compared with 0/26 (0%) HPV+ CIN 0/I controls and 3/95 (3%) HPV+ CIN II/III. MFI ratios of individual HPV16 serology were similar in women with CIN 0/I and women with CIN II/III.

No significant correlation between HPV16L1 bead array signal intensities, cLIA VLP titer, and pseudovirion assay were observed, likely representing display of different antigenic structures detected in the two assays (data not shown). A total of 66 patient samples were assayed by the cLIA and PsVN assays, and 31 were CIN 0/I and 35 were CIN II/III. Sixteen CIN 0/I patients were cLIA+, of which 13 were also PsVN+. All the cLIA+ were HR HPV+, but only 13 were HPV16+. Thirty of the CIN 0/I were L1− by the bead ELISA. Of the CIN II/III group, 20 patients were cLIA+, with 19 also being PsVN+ and HR HPV+. Five of the 19 were also L1+ by the bead ELISA. Our L1 assay correlates with a small subset (5/35) of PsV+ CIN II/III, with only 1 discordant (L1 Ab+/PsV−) case, and does not correlate with the Merck cLIA L1 assay.

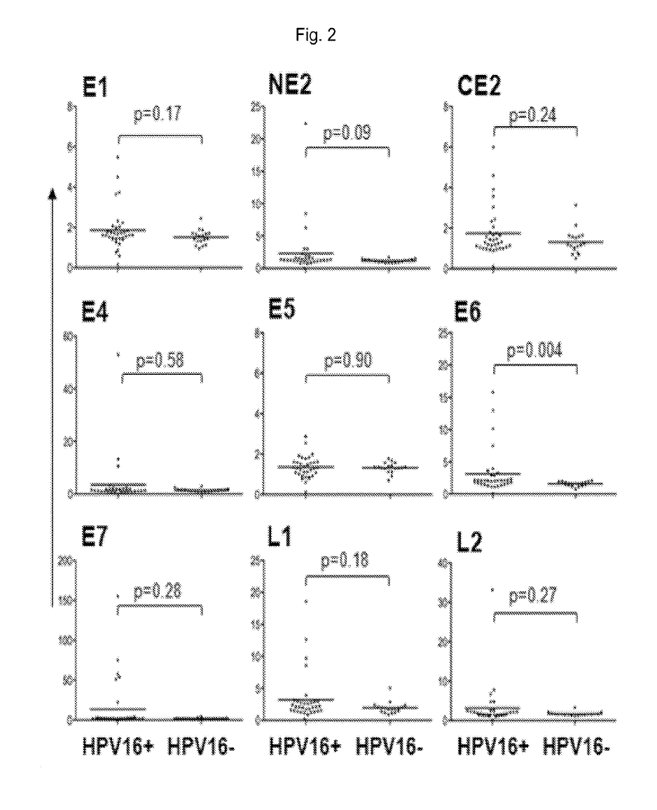

To compare the frequency of HPV16 Abs in sera from patients with CIN II/III and ICC, blood from 95 patients with invasive cervical carcinoma were examined for HPV16 antibodies (FIG. 1). With invasive cervical neoplasia, E1, NE2, E4, E5, E6, E7, and L1 Ab levels increase (p<0.0001). To determine if the detection of HPV16 antibodies was specific for HPV16+ ICC, we performed subset analysis of the samples from women with cervical tumors known to be HPV16+ ICC cases (n=34, of which 4 had multiple infections that included HPV16) and those with tumors known to be HPV16-negative controls (n=20) (FIG. 2a). Forty-one ICC tumors had unknown HPV-infection status and these sera were excluded from the analysis. We confirmed that HPV16E6 Abs were specifically detected in the blood of patients with HPV16+ ICC. Abs to E1, NE2, E4, E6, E7, L1, and L2 were selectively detected in HPV16+ ICC cases (13/34 (39%) cases had at least one early-gene Ab).

To determine whether HPV16-specific Abs were secreted in the cervix, cervical secretions were collected using cervical swabs. We directly compared the detection of HPV16-specific antibodies in sera and in cervical secretions from 87 patients with CIN 0/1, CIN II/III and ICC (data not shown). For ICCs, there was a strong correlation between detection of IgG in cervical secretions and the sera (R²=0.73-0.99), but cervical IgG was, on average, weaker than serum IgG. Since IgA may have higher concentrations in secretions than IgG, we directly compared the detection of HPV16-specific IgG and IgA antibodies in sera (not shown) and cervical secretions from patients with ICC (FIG. 2b). IgG detection was stronger than IgA for E6 (p=0.0004), E7 (p=0.02), and L1 (p=0.007). There was no specific detection of IgA responses to other HPV16 antigens, or in CIN II/III (data not shown). These results suggest that there is no benefit to measuring mucosal IgG or IgA over serum IgG, although mucosal IgG may have clinical utility as a non-invasive assay.

One concern about a serum assay for HPV Abs is the emerging prevalence of extra-cervical HPV-related malignancies, such as HPV+ OPC. We recently identified strong HPV16 E1, E2, E4, E6, and E7 Absinthe sera of newly diagnosed OPC patients. The detection of E1 and E2 Abs in patient sera was surprising, since that had not been identified in ICC. Here, we directly compared HPV16 Ab levels from patients with invasive cervical cancers (n=95, of which 34 (36%) were known HPV16+) and OPC (n=50 known HR HPV+, of these 50,28 (56%) were known HPV16+ and >90% of the remaining were estimated to be HPV16+).

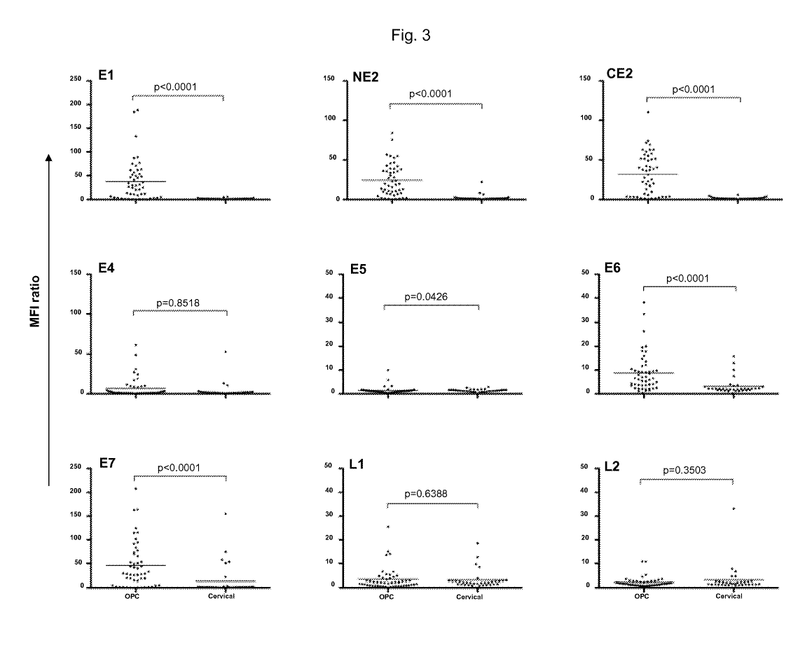

As expected, the OPC cases were older than the patients with cervical cancer (mean 54.7 yrs) and were predominantly male, consistent with clinical incidence. We have identified no differences in HPV16 Ab levels between male and female OPC patients (data not shown). These were retrospective samples, and ICC and OPC sera were collected from different regions of the country. Abs detected by the bead array were significantly higher in invasive OPC sera than in invasive cervical sera (FIG. 3).

In particular, several Abs discriminated between ICC and OPC sera: E1, NE2, CE2, E6, and E7 (p<0.001), suggesting that E1 and E2 Abs may be specific biomarkers for OPC. As seen in FIG. 3, strong E7-specific Abs responses were detected in 6/34 (18%) HPV16+ ICC cases, suggesting that these patient were capable of mounting IgG responses to early-gene Ab. None of those cases had detectable E1 or E2 Abs. In contrast, 92% of the HR HPV+ OPC cases with E7 Abs had E1 and/or E2 Abs. As expected, there was no significant difference in L1/L2 serology (which represents response to productive infection) between OPC and ICC cases.

To evaluate the additive benefit of HPV16 Ab detection with HR HPV typing to identify cases of CIN II/III in a colposcopy clinic setting, we performed a multivariate analysis to determine if HPV16 Abs increased the specificity of detection of CIN II/III. Cut-off values for positive serology for each HPV16 antigen were established using sera from 50 healthy donors of unknown HPV or exposure status (Table 2b). Sera from subjects with CIN 0/I and CIN II/III had low frequency of antibody detection to any of the HPV16 antigens (0-4%) in this assay. 6/121 (5%) of CIN 0/I controls and 13/162 (8%) of CIN II/III cases were positive for at least one antibody. Using all Abs without HR HPV status, we developed a logistic regression classifier with a sensitivity of 95.7% but a specificity of only 20.7%; in a leave-one-out cross-validation study, the classifier yielded 92.0% sensitivity and 17.4% specificity. Overall, addition of antibody levels to HR HPV DNA typing resulted in modest improvement in specificity at lower sensitivities for the detection of CIN II/III.

Of the subset of ICC cases that were confirmed HPV16+, 6/34 cases (18%) were positive for E7-Abs, 6/34 (18%) were positive for E4-Abs, and 11/34 (32%) were positive for either E4- or E7-Abs (Table 2b). For HR HPV+ OPC cases (estimated >90% HPV16+), 36/50 (72%) were positive for each of E1, NE2, CE2, and E7, with lower frequency of detection of E4-Abs or E6− Abs (40%). Multiplexed assessment of all early gene Abs improved the sensitivity of detection, with 21195 (22%) of ICC cases, 13/34 (38%) of ICC HPV16+ cases, and 47/50 (94%) of HR HPV+ OPC cases positive for at least one antibody.

A logistic regression classifier based on all early gene Abs further improved specificity of detection for OPC compared to cervical disease, yielding positivity in 1195 (1%) of ICC cases, 1/34 (2.9%) of iCC HPV16+ cases, and 46/50 (92%) of HR HPV+ OPC cases, compared with 4/121 (3%) of CIN 0/I controls and 11/162 (7%) of CIN II/III cases. In a cross-validation study, the classifier yielded positivity in 2/95 (2%) of ICC cases, 2/34 (5.9%) of iCC HPV16+ cases, 44/50 (88%) of HR HPV+ OPC cases, 4/121 (3%) of CIN 0/1 controls and 11/162 (7%) of CIN II/III cases.

Current screening guidelines for cervical disease relies on primary cytologic screening, such as the Papanicolaou (Pap) test for routine screening of women over the age of 21. The sensitivities of Pap test are limited (estimated at 51-61%), possibly higher with the liquid-based cytologic testing, which is more commonly used in the United States. The incorporation of molecular assays, such as HPV DNA testing, into screening strategies for the detection of pre-invasive cervical disease is a subject of several recent large-scale clinical trials. These results demonstrated that HPV DNA testing is significantly more sensitive than cytologic screening for high-grade CIN, but has a lower specificity. In the randomized Canadian Cervical Cancer Screening Trial, Pap test screening had a sensitivity of 55.4% compared with HPV DNA testing (94.6%) for women over age 30. Similarly, in a randomized controlled trial from Sweden, the combination of HPV DNA and Pap test screening resulted in a reduction in the incidence of CIN II/III, but ongoing double screening led to both increased cost and reduced positive predictive values of women referred for colonoscopy. Serial co-testing with liquid-based cytologic screening and HPV DNA did not lead to a significant change in detection of CIN II/III, but is now in clinical use in the U.S. for women over 30. Development of biomarkers that improve the specificity of HPV DNA testing for the diagnosis of CIN II/III could markedly impact the utility of HPV DNA testing as a primary screening tool.

The development of effective HPV vaccines targeting HPV16 and HPV18 is predicted to alter the pre-test probability of HPV-targeted screening assays. The impact of vaccines on cancer incidence will not occur for more than 15 years after achieving high coverage because of the long natural history between infection and neoplasia. Screening for vaccine-missed cervical cancers will require even more efficient cost-effective and specific screening tools as the vaccines will have a greater impact on high grade lesions that require treatment than on low-grade lesions that result in most referrals for follow-up.

Here, using a novel assay for the detection of HPV16-specific IgG Abs in human sera, we measured the frequency of HPV16-specific early gene Abs in sera of a cohort of women referred for colposcopy. Our results demonstrate that Abs to multiple HPV16-derived early proteins were not specifically detected in the sera of untreated patients with CIN II/III, compared with CIN 011 controls and insensitive to infection without invasive cancer. While HPV DNA may detect transient, as well as persistent HPV infections, it is likely that HPV-specific early gene Abs develop only after persistent infection or the development of cancer. We focused on the detection of HPV16-specific Abs, which are detected in up to 55% of cervical cancers. HPV16-specific antibodies to E1, E2, E4, E6, and E7 were detected in invasive cervical disease, with a prevalence of 38% of HPV16+ cases (22% overall) positive for at least one antibody.

For invasive disease, detection of HPV16-specific Abs was specific for cases with HPV16 or HR HPV DNA by PCR. While extension of the serologic assay to other oncogenic HPV types may improve the sensitivity and utility of the Ab assay for invasive cancer, it is unlikely to be of use for selection of high risk patients for colposcopy, but may have utility for the early detection of invasive cervical cancer.

The emergence of HPV16 as a major and dominant risk factor for oropharyngeal cancers raises the concern that detection of HPV-specific early gene serum Abs will not distinguish between cervical disease and oropharyngeal disease, resulting in double referrals to colonoscopy and otolaryngology clinics. It is not known if prophylactic vaccines will prevent HPV-associated oropharyngeal tumors, and direct demonstration in clinical trials is difficult because of the lack of well-recognized cancer precursors that could be used as surrogate endpoints. Comparing the immune response to HPV proteins in persons with HPV-positive tumors in cervix versus the oropharynx could provide indirect evidence of similarities or differences in the mechanisms of host/viral interactions and the pathogenesis of cancer in these two different anatomic sites. This may also identify patients at high risk for OPC, for early intervention.

Here, we directly compared HPV16 Ab detection between patients with invasive cervical and invasive oropharyngeal cancers. We detected a striking difference between the strong E1- and E2-specific Abs detected in the sera of patients with OPC, which was not detected in patients with invasive cervical disease. This may explain why these Abs have not been described even by viral-proteome screening in cervical cancer, although differences in protein display of antigenic structures may be minor contributing factors. Although there were differences in both age and gender between the invasive cervical and OPC patients tested here, detection of Abs to E1/E2 was unrelated to age or gender in OPC.

A direct comparison of HPV viral transmission, natural history and pathogenesis between OPC and cervical cancer has not yet been performed. Clear demonstration of viral DNA and viral oncoproteins in OPC tumors suggest the same pathogenetic mechanisms may occur. Since E1/E2 genes are disrupted by integration of the virus, and Ab detection usually depends on antigen expression, our data suggests there are differences in the rates of expression of E1/E2 and possibly viral integration and episomal forms between OPC and cervical cancer suggesting that these two entities may be different both clinically and biologically and this may impact HPV-targeted therapies.

TABLE 1
Characteristics of Study Samples

| | Disease Status | | | |
|---|---|---|---|---|
| | CIN 0/I | CIN II/III | ICC | HPVOPC |
| Charac- | N = 121 | N = 162 | N = 95 | N = 50 |
| teristics | N (%) | N (%) | N (%)* | N (%)* |
| Age in yrs, | 29.1 (9.5) | 29.3 (8.4) | 50.1 (14.7) | 54.7 (8.1) |
| Mean | | | | |
| <30 | 76 (62.8) | 94 (58.0) | 8/91 (8.8) | 0 (0) |
| ≧30 | 45 (37.2) | 68 (42.0) | 83/91 (91.2) | 49/49 (100.0) |
| Race | | | | |
| Black | 99 (81.8) | 100 (61.7) | 65/91 (71.4) | 1/49 (2.0) |
| Other | 22 (18.2) | 62 (38.3) | 26/91 (28.6) | 48/49 (98.0) |
| Sex | | | | |
| Female | 121 (100) | 162 (100) | 95 (100) | 3/49 (6.1) |
| Male | 0 | 0 | 0 | 46/49 (93.9) |
| HPV16 | | | | |
| DNA | | | | |
| status | | | | |
| HPV16+ | 26 (21.5) | 95 (58.6) | 34/67 (50.7) | |
| HPV DNA | | | | |
| status | | | | |
| overall | | | | |
| Negative | 37 (30.6) | 5 (3.1) | 13/67 (19.4)^{†} | |
| 1 HPV type | 41 (33.9) | 78 (48.1) | 49/67 (73.1)^{†} | |
| 2 HPV types | 22 (18.2) | 38 (23.5) | 3/67 (4.5)^{†} | |
| ≧3 HPV | 21 (17.4) | 41 (25.3) | 2/67 (3.0)^{†} | |
| types | | | | |
| Any HR | 71 (58.7) | 155 (95.7) | 54/67 (80.6)^{†} | 50 (100) |
| HPV^{‡} | | | | |

*N varies for each category because of missing information.
^{†}HPV testing methods used for anonymized archived samples differed from those used in biorepository, so results are not directly comparable.
^{‡}The following HPV types were considered as high risk types for this analysis -HPV16, 18, 31, 33, 35, 39, 45, 51, 52, 56, 58, 59, 66, 68

TABLE 2a
MFI ratios for HPV16 antibodies stratified by diagnosis.

| HPV16 | Median fluorescence intensity (MFI) ratio* (range) | | | | |
|---|---|---|---|---|---|
| Abs | CIN 0/I | CIN II/III | ICC | ICC HPV16+ | HPVOPC^{‡} |
| E1 | 1.1 (0.1-4.3) | 1.1 (0.1-3.0) | 1.7 (0.2-5.5) ^{†} | 1.9 (0.2-5.5) ^{†} | 37.5 (0.3-188.7) ^{§} |
| NE2 | 0.9 (0.1-4.3) | 1.1 (0.2-8.2) | 1.6 (0.1-22.4) ^{†} | 2.3 (0.1-22.4) ^{†} | 24.3 (0.3-84.2) ^{§} |
| CE2 | 1.9 (0.4-5.8) | 2.1 (0.4-7.9) | 1.4 (0.1-6.0) ^{†} | 1.7 (0.1-6.0) | 31.7 (0.8-110.5) ^{§} |
| E4 | 1.1 (0.3-15.0) | 0.9 (0.2-4.9) | 2.7 (0.1-52.9) ^{†} | 3.6 (0.1-52.9) ^{†} | 6.8 (0.3-61.9) |
| E5 | 1.0 (0.2-3.1) | 1.0 (0.2-3.6) | 1.4 (0.1-2.9) ^{†} | 1.4 (0.1-2.9) ^{†} | 1.4 (0.3-10.1) |
| E6 | 1.4 (0.3-17.6) | 1.7 (0.2-20.0) | 2.2 (0.1-15.8) ^{†} | 3.1 (0.1-15.8) ^{†} | 8.8 (0.4-38.2) ^{§} |
| E7 | 1.2 (0.4-4.9) | 1.4 (0.4-13.9) | 8.3 (0.1-212.4) ^{†} | 13.6 (0.1-155.0) ^{†} | 45.7 (0.5-207.7) ^{§} |
| L1 | 1.6 (0.1-16.0) | 2.1 (0.1-17.1) | 2.3 (0.2-18.6) ^{†} | 3.2 (0.2-18.6) ^{†} | 3.4 (0.2-25.5) |
| L2 | 1.7 (0.5-10.7) | 1.9 (0.5-11.9) | 2.2 (0.2-33.1) ^{†} | 3.1 (0.2-33.1) ^{†} | 2.2 (0.7-10.9) |

*MFI ratio of HPV-GST antigen/p21-GST
^{†} Compared to CIN 0/I, p < 0.005 using unpaired Wilcoxon, bold
^{‡}High Risk HPV cases only
^{§} Compared to ICC, p < 0.005 using unpaired Wilcoxon, bold

TABLE 2b
Prevalence of positive antibody response* to each HPV16 protein stratified by diagnosis.

| | Samples Positive for HPV16 Abs | | | | | | |
|---|---|---|---|---|---|---|---|
| | CIN 0/I #(%) | | CIN II/III #(%) | | ICC #(%) | | HPVOPC^{†} #(%) |
| HPV16 | Total | HPV16+ | Total | HPV16+ | Total | HPV16+ | Total |
| Abs | n = 121 | n = 26 | n = 162 | n = 95 | n = 95 | n = 34 | n = 50 |
| E1 | 0 (0%) | | 0 (0%) | | 1 (1%) | 1 (3%) | 36 (72%) |
| NE2 | 0 (0%) | | 0 (0%) | | 1 (1%) | 1 (3%) | 36 (72%) |
| CE2 | 0 (0%) | | 2 (1%) | 1 (1%) | 0 (0%) | | 36 (72%) |
| E4 | 4 (3%) | 1 (4%) | 3 (2%) | 2 (2%) | 12 (13%) | 6 (18%) | 20 (40%) |
| E5 | 0 (0%) | | 0 (0%) | | 0 (0%) | | 2 (4%) |
| E6 | 1 (1%) | | 3 (2%) | 2 (2%) | 3 (3%) | 3 (9%) | 20 (40%) |
| E7 | 0 (0%) | | 2 (1%) | | 8 (8%) | 6 (18%) | 36 (72%) |
| L1 | 1 (1%) | | 7 (4%) | 4 (4%) | 3 (3%) | 3 (9%) | 4 (8%) |
| L2 | 2 (2%) | 1 (4%) | 3 (2%) | | 2 (2%) | 1 (3%) | 2 (4%) |
| E1, E2, | 1 (1%) | | 6 (4%) | 3 (3%) | 11 (12%) | 9 (26%) | 46 (92%) |
| E6 or E7^{‡} | | | | | | | |

*Cut-off values defined as average MFI ratio +3 standard deviations for each antigen in serum of healthy controls
^{†}HR HPV cases only
^{‡}Positive for at least one of E1, NE2, CE2, E6, and E7

### Example 2

Using a custom bead array ELISA, Abs to the HPV16 proteome were measured in sera from patients with no cervical disease (CIN 0, n=33), high-grade cervicaldysplasia (CINIII/III, n=52), invasive cervical carcinoma (ICC, n=13), and oropharyngeal cancer (OPC; n=15). The median fluorescent intensity (MFI) ratios of IgG specific for each HPV-GST antigen to control p21-GST antigen were determined. All cervical cases and controls were typed for HPV DNA by Roche linear array.

In comparison to CIN0, CINII/III sera had an increased MFI ratio of Abs to HPV16 E2, E6, and E7 (p<0.05). There were no detectable differences in Abs to E1, E4, and E5 for CINII/III. There was a trend to the detection of L1 and L2 specific Abs in CINII/III (p=0.1). In comparison, sera from 4/13 patients with ICC had detectable Abs E4, E6, and/or E7. OPC sera had significantly higher MFI ratios of Abs to E1, E2, E6, and E7 antigens than ICC patients (pS0.05).

Conclusions: Serum Ig G Abs to HPV 16 E2, E6, and E7 proteins are detected in high grade cervical dysplasia. The response broadens in ICC; in contrast to ICC, OPC sera contain strong E1 and E2 Abs, suggesting differences in biology reflected in the antibody responses between these two HPV-associated malignancies.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detection of human papillomavirus (HPV) mediated cervical or oropharyneal cancer, comprising the steps of:
contacting a fluid sample from a patient with multiple antibodies to HPV16 early gene proteins; and
comparing patterns of HPV16 antibody bound to said early gene proteins with a control associated with cervical or oropharyneal cancer.

2. The method of claim 1, wherein said early gene proteins comprise E1, E2, E4, E6, E7, L1, and L2.

3. A method for diagnosing high grade cervical dysplasia versus invasive cervical cancer, comprising the steps of:
contacting a fluid sample from a patient with multiple antibodies to HPV16 early gene proteins; and
comparing patterns of HPV16 antibody bound to said early gene proteins with a control associated with high grade cervical dysplasia versus invasive cervical cancer.

4. The method of claim 3, wherein said multiple antibodies comprise Serum IgG antibodies to HPV 16 E2, E4, E6, and E7 proteins.

5. A method for diagnosing oropharyneal cancer, comprising the steps of:
contacting a fluid sample from a patient with multiple antibodies to HPV16 early gene proteins; and
comparing patterns of HPV16 antibody bound to said early gene proteins with a control associated with oropharyneal cancer.

6. The method of claim 5, wherein said multiple antibodies comprise Serum IgG antibodies to HPV 16 E1 and E2.

7. A system for diagnosing human papillomavirus (HPV) mediated cancer, comprising:
a substrate having multiple antibodies to HPV16 early gene proteins coupled thereto; and
a visualization agent; and
a control with a binding pattern associated with a HPV-mediated cancer for comparing visualized patterns of HPV16 antibody bound to said early gene proteins with said control associated with said HPV-mediated cancer.
